(19) United States
(12) Patent Application Publication
(10) Pub. No.: US 2019/0133799 A1
(43) Pub. Date: May 9, 2019

(54) BALLOON EXPANDABLE, BIOABSORBABLE, DRUG-COATED SINUS STENT

(71) Applicant: Biovention LLC, El Monte, CA (US)

(72) Inventors: Ella Yongxi LIANG, Claremont, CA (US); Yongwei CHIEN, Suzhou (CN); Ye ZHOU, Suzhou (CN)

(21) Appl. No.: 15/976,227

(22) Filed: May 10, 2018

(30) Foreign Application Priority Data
Nov. 8, 2017 (CN) 201711092696.5

(51) Int. Cl. A61F 2/958; A61L 31/08; A61L 31/14; A61F 2/94

(52) U.S. Cl. CPC A61F 2/958; A61L 31/082; A61L 31/022; A61F 2/94; A61L 31/148

(57) ABSTRACT

This invention discloses a balloon expandable, bioabsorbable, drug-coated sinus stent, including sinus stent delivery system and stent, the sinus stent delivery system is composed of conical balloon and push shaft, the sinus stent delivery system is for delivering and expanding the fixed stents. The stent is made of biodegradable material, and its surface is coated with a biodegradable and expandable anti-inflammatory drug protective film. The shape of the stent is cylindrical; The stent is conical after expansion by the balloon. The external wall of the stent is composed of a wavy net structure.

### FIELD OF THE INVENTION

The invention relates to a system device used to treat the blockage of a nasal cavity, belonging to the medical devices field, and in particular to a Balloon Expandable, Bioabsorbable, Drug-Coated Sinus Stent.

### BACKGROUND

Sinusitis and allergic rhinitis are common and frequent cases, occurring between 5 and 79 years old people. Survey by the department of public health: the global rates as high as 10%-14% of allergic rhinitis, sinusitis incidence is about 15% of the population, nearly 600 million people worldwide are suffering from rhinitis of harassment, and have been gradually rising.

66 percent of asthmatics are victims of allergic rhinitis. As experts recommended, more than ⅓ of patients with allergic rhinitis develop into asthma if not properly treated.

The pathological dilatation, such as sinus sinusitis balloon dilatation treatment after a period of time, sinus lesions symptoms will narrow again, lead to recurrent lesions, which needed a second operation and repeated examination. It increases the patient's pain and financial burden.

In a Chinese invention patent CN 106955175 A, public A medical stent had made public, the stent is made of the poly lactic acid polymer or biological absorbable magnesium alloy, and the surface coated with A layer of biodegradable antiphlogistic drug protective film, the design of the protective film with anti-inflammatory drugs make the stents can not only recover narrow nasal cavity, and to diminish inflammation lesion area. However, the stent was installed in the nasal cavity and to fix it in the lesion by inflation, and the stent was propped up to the cylinder to dredge the nasal cavity. However, the above medical stent has a displacement slip during fixation in the nasal cavity, which affects the recovery of the nasal cavity.

### SUMMARY OF THE INVENTION

In order to overcome the above disadvantages, the purpose of the present invention is to provide a medical stent that is not easy to shift and slide after being installed into the lesion in the nasal cavity.

In order to achieve the above objectives, the technical solution adopted for the invention is: a balloon expandable, bioabsorbable, drug-coated sinus stent, including sinus stent delivery system and stent, the sinus stent delivery system is composed of conical balloon and push shaft, the sinus stent delivery system is for delivering and expanding the fixed stents. The stent is made of biodegradable material, and its surface is coated with a biodegradable and expandable anti-inflammatory drug protective film. The shape of the stent is cylindrical; The stent is conical after expansion by the balloon. The external wall of the stent is composed of a wavy net structure.

As preferred, the conical balloon diameter is 1-20 mm, length is 5-100 mm, both ends diameter difference value is 0.5-2 mm, it's benefit for stent to be fixed in the sinus and not to slip out.

As preferred, the wavy net structure is coiled around the central axis of the stent with a number of parallel wave wires, and the connecting wires are arranged between the two connected wave wires. Through the wavy assembly, increase the stent strength and reduce the usage of relevant material, thereby reduced the cost (wavy structure itself has certain elasticity, and can have very good supporting).

As preferred, the stent wall thickness is 0.1-5.00 mm. This thickness is suitable for the diseased parts of the body.

As preferred, the material of anti-inflammatory drug protective film is mometasone furoate or fluticasone propionate. The biodegradable materials are polylactic acid polymers or bioabsorbable magnesium alloys.

As preferred, the stent length is 5-100 mm, expanded diameter is 1-20 mm. This size is suitable for the diseased region.

As preferred, the conical expansion state is the circular structure. The improvement of this shape is conducive to industrial production.

### BRIEF DESCRIPTION OF THE DRAWING

FIG. 1 The main view before the expansion of the Bioabsorbable, Drug-Coated Sinus Stent;

FIG. 2 The side view before the expansion of the Bioabsorbable, Drug-Coated Sinus Stent;

FIG. 3 The main view after the expansion of the Bioabsorbable, Drug-Coated Sinus Stent;

FIG. 4 The side view after the expansion of the Bioabsorbable, Drug-Coated Sinus Stent;

FIG. 5 The main view of the sinus stent delivery system.

In fig:

1—stent; 2—wavy net structure; 3—anti-inflammatory drug protective film; 4—balloon; 5—push shaft; 6—sinus stent delivery system.

### DESCRIPTION OF ILLUSTRATIVE EMBODIMENTS

Below is detail implementation example description in combination with the appended drawings, so that the advantages and characteristics of the present invention can be easy to understand, so this invention is clearly definited of the scope of protection.

### Implementation Example

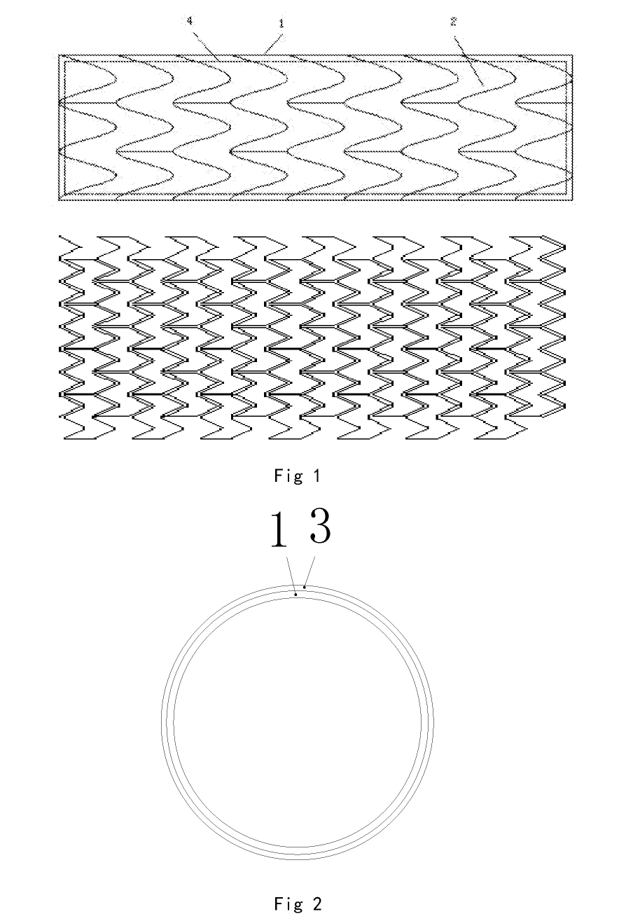

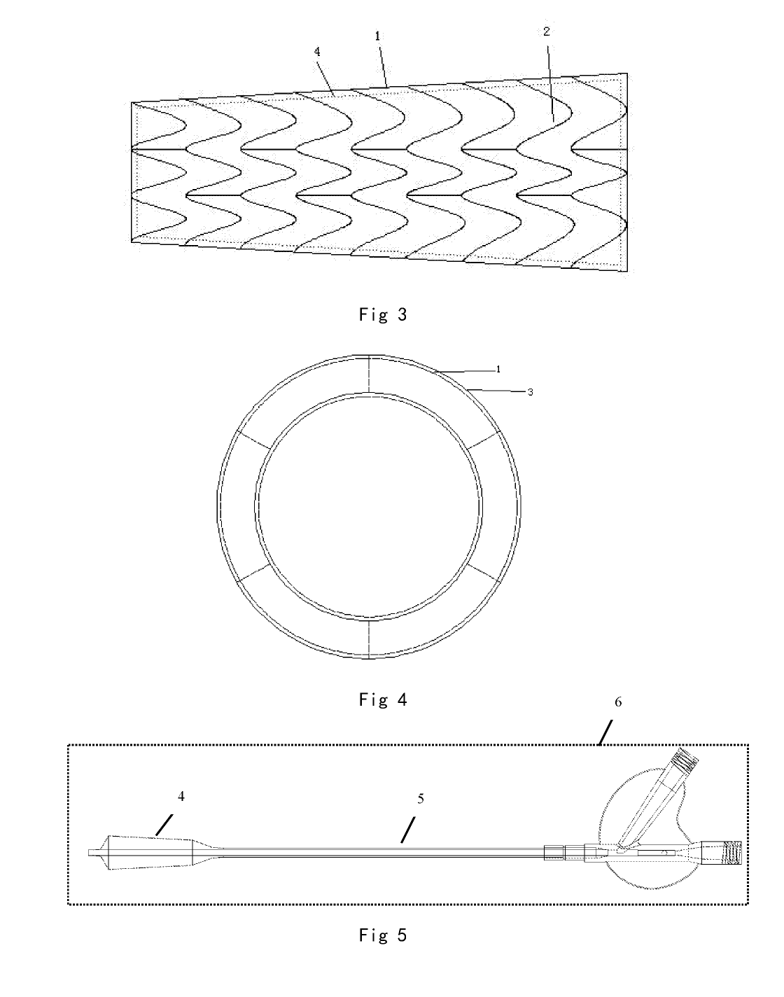

See attached FIG. 1-5, in this example, A Balloon Expandable, Bioabsorbable, Drug-Coated Sinus Stent, including stent 1, the length of the stent 1 is 5-100 mm, stent 1 wall thickness is 0.1-5.00 mm. The stent 1 is made of biodegradable materials such as poly (lactic acid) polymers or bioabsorbable magnesium alloys. Stent 1 has internal tune, internal tune is fixed on balloon 4 of the stent delivery system 6, stent 1 and balloon 4 fixed by crimping, balloon 4 connected with an inflatable and deflatable push shaft 5 to connect to the external air tube.

Stent 1 can be delivered to lesion region by the sinus stent delivery system.

When the balloon 4 of the sinus stent delivery system inflated by the pushshaft 5, the stent 1 will be expanded to conical expanded state, and can be well fixed at lesion region, increased the stability of the stent fixing. When balloon 4 deflated, the stent 1 fixed at lesion region and the balloon 4 will be taken out the body together with the push shaft 5. In this implementation example the conical expansion state is circular structure. The improvement of this shape is conducive to fixing and not easy to slip out and industrial production.

In order to reduce the relevant material with the same strength, the stent 1 is with a wavy net structure 2, the wavy net structure 2 is coiled around the central axis of the stent 1 with a number of parallel wave wires, and the connecting wires are arranged between the two connected wave wires. Through the wavy assembly, increase the stent strength and reduce the usage of relevant material, thereby reduced the cost (wavy structure itself has certain elasticity, and can have very good supporting).

The invention can support the lesion region at least twice the time by expanding the stent in the lesion stenosis with the balloon 4 expansion. The shape of the biodegradable stent before expanding is cylindrical, and the expansion diameter was enlarged and the diameter was 1-20 mm, and the whole structure was round.

Stent 1 is coated with a layer of biodegradable, expanding antiphlogistic drug protective film 3, anti-inflammatory drugs protective film 3 can use mometasone furoate or fluticasone propionate, the drug need to be mixed with poly lactic acid polymer, mix proportion ranging from 10% to 95%, according to the specific requirements.

In usage, the stent 1 will be fixed on the balloon 4 of the sinus stent delivery system 6 by crimping, when balloon 4 get to sinus lesion region, inflate the balloon 4, the stent 1 will expansion, deflate the balloon 4, Bioabsorbable, Drug-Coated Sinus Stent fixed in the lesion region and support the narrow area; As the stent 1 is degradable, the stent 1 no need to remove from the lesion region, the stent 1 and anti-inflammatory drugs protective film 3 stay in lesion region, after a certain time, the stent 1 and anti-inflammatory drugs protective film 3 will degradate, and anti-inflammatory drugs protective film 3 can diminish inflammation, to reduce the likelihood of reoperation, no need to anti-inflammatory drugs in the affected area, reduce patients pain, with only one operation.

In this invention, the stent shape changed from the original cylinder into conical expansion state, in installation the expanded site first entrance into lesion area, when inflated, the stent expanded and fixed at lesion area more stable and cover the problem of stent slipping out.

Above implementation methods of this invention are for technical conception and characteristic, the purpose is to let people know well the technology and to understand the content of this invention to implementation, and it cannot limit the scope of protection of the invention, all the present invention spirit of equivalent change and modify, should be covered within the scope of protection of this invention.

What is claimed:

1. A balloon expandable, bioabsorbable, drug-coated sinus stent, including sinus stent delivery system and stent, its characteristic is: including the sinus stent delivery system 6 and stent 1, the sinus stent delivery system 6 is composed of conical balloon 4 and push shaft 5, the stent 1 is made of biodegradable material, and its surface is coated with a biodegradable and expandable anti-inflammatory drug protective film 3, the balloon 4 is connected to a push shaft 5 which can connect to an external air tube, before balloon 4 inflated, the shape of the stent is cylindrical; the stent 1 is conical after expansion by the balloon 4, and the external wall of the stent 1 is composed of a wavy net structure.

2. According to claim 1 the bioabsorbable, drug-coated sinus stent, its characteristic is: the balloon 4 is conical, the diameter is 1-20 mm, the length is 5-100 mm, the both end diameter difference value is 0.5-2 mm.

3. According to claim 1 the bioabsorbable, drug-coated sinus stent, its characteristic is: the stent 1 before expanding is cylindrical.

4. According to claim 1 the bioabsorbable, drug-coated sinus stent, its characteristic is: the stent 1 is with a wavy net structure, the wavy net structure is coiled around the central axis of the stent 1 with a number of parallel wave wires, and the connecting wires are arranged between the two connected wave wires.

5. According to claim 4 the bioabsorbable, drug-coated sinus stent, its characteristic is: The reticular or hollow structure (2) is weaved or cutted.

6. According to claim 4 the bioabsorbable, drug-coated sinus stent, its characteristic is: the stent wall thickness is 0.1-5.00 mm.

7. According to claim 1 the bioabsorbable, drug-coated sinus stent, its characteristic is: the material of anti-inflammatory drug protective film 3 is mometasone furoate or fluticasone propionate, the mix proportion ranging from 10% to 95%; the biodegradable materials are polylactic acid polymers or bioabsorbable magnesium alloys.

8. According to claim 7 the bioabsorbable, drug-coated sinus stent, its characteristic is: the stent 1 length is 5-100 mm, expanding diameter is 1-20 mm.

9. According to claim 1 the bioabsorbable, drug-coated sinus stent, its characteristic is: the conical state is the circular structure.
